(19) United States
(12) Patent Application Publication
(10) Pub. No.: US 2023/0338181 A1
(43) Pub. Date: Oct. 26, 2023

(54) ORTHOSIS AND METHOD OF USE

(71) Applicant: NEOFF MEDICAL PTY LTD, Northcote (AU)

(72) Inventor: Geoffrey DOWLING, Northcote (AU)

(73) Assignee: NEOFF MEDICAL PTY LTD, Northcote (AU)

(21) Appl. No.: 18/025,573

(22) PCT Filed: Aug. 18, 2021
(86) PCT No.: PCT/AU2021/050911

§ 371 Date: Mar. 9, 2023

(30) Foreign Application Priority Data
Sep. 10, 2020 (AU) 2020903244

(51) Int. Cl. A61F 5/14; A43B 7/24; A43B 7/22; A43B 7/1475; A43B 17/00

(52) U.S. Cl. CPC A61F 5/14; A43B 7/24; A43B 7/223; A43B 7/1475; A43B 17/00

(57) ABSTRACT

An orthosis for footwear includes an insert having a plurality of retainers, and a plurality of adjustment elements of predetermined configuration which can be removably retained by the insert to alter the shape of the insert adjacent the arch of a subject's foot when located in the footwear. The orthosis provides an improved device and method for alleviating at least one of the symptoms of a foot condition, such as flat feet.

### CROSS-REFERENCE TO RELATED APPLICATIONS

This application is a national phase entry of PCT Patent Application Serial No. PCT/AU2021/050911 filed on Aug. 18, 2021, which claims priority to Australian Patent Application Serial No. 2020903244 filed Sep. 10, 2020, both of which are incorporated by reference herein.

### TECHNICAL FIELD

The present invention relates to the field of orthotics, particularly orthoses for foot correction. In one form, the invention relates to a foot orthosis in the form of a shoe insert. In one particular aspect the present invention is suitable for use by a subject having pes planus which is a condition more commonly known as “flat feet” or “fallen arches”. It will be convenient to hereinafter describe the invention in relation to flat feet, however it should be appreciated that the present invention is not limited to that use only and may have wider application to other foot disorders such as those with a pes cavus foot posture more commonly known as a “high arch” or conditions in other parts of the body caused by a foot condition or foot alignment.

### BACKGROUND

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

Orthotics focuses on the design and fitting of external devices to modify the structural and functional characteristics of the neuromuscular and skeletal system. For example, an orthosis may be applied to an extremity, joint or other part of the body to:
Guide, limit or immobilize;
Assist movement generally;
Reduce weight bearing forces;
Aid rehabilitation after injury;
Adjust shape or function;
Reduce pain or other discomfort.

Design of orthotics requires knowledge of anatomy, physiology, pathophysiology, biomechanics and engineering. Foot orthoses are devices inserted into shoes to provide support for the foot by redistributing ground reaction forces acting on the foot joints while a subject is standing, walking or running. Foot orthoses may also indirectly assist with correcting problems associated with the knee, hip and spine.

Foot orthoses are effective at reducing pain and discomfort caused by alignment related issues such as flat feet and high arched foot posture. They are also effective at addressing overuse injuries such as but no limited to plantar fasciitis and Achilles tendinopathy. Foot orthoses may also be used in conjunction with properly fitted orthopaedic footwear for subjects prone to suffering foot ulcers such as diabetes.

Some foot orthoses are mass produced and are typically sold by local retailer. For example, A. Alego Pty Ltd provides Interpod EVA Modular Orthoses (website located at https://www.algeos.com.au/Interpod_Modular.html) which consists of a base orthotic that can be inserted along three quarters of the length of a shoe. The base orthotic can receive a low, moderate or high insert that runs the entire length of the base orthotic from the heel to the toe region, providing an even arch profile.

Stable Step, LLC provides Powerstep ProTech Custom Post (website located at https://www.powersteps.com/protech-custom-post.html) orthotic inserts comprising a shell which can be adjusted by heel lift inserts or varus and valgus heel wedge inserts. The posts and heel heights snap into the base of the shell and stay securely in place as described in U.S. Pat. No. 7,913,429.

Many orthoses are custom made. In the past, custom made orthoses were created based on foot tracings and measurements. Subsequently, plastic has become widely used for orthoses, the plastic being conformed to a plaster of Paris mould of the subject's foot. In more recent times CAD/CAM, CNC machines and 3D printing have been applied to creations and manufacture of orthoses. Orthoses are made from various types of materials including thermoplastics, carbon fibre, metals, elastic, EVA, fabric or a combination of similar materials.

Flat Feet (Also Known as Pes Planus or Fallen Arches)

The condition known as “flat feet” is a postural deformity in which the arches of the foot collapse, with the entire sole of the foot coming into complete or near-complete contact with the ground. There is a functional relationship between the structure of the arch of the foot and the biomechanics of the lower leg. The arch provides an elastic, springy connection between the forefoot and the hind foot so that a majority of the forces incurred during weight bearing on the foot can be dissipated before the force reaches the long bones of the leg and thigh.

When a patient has a flat feet, the head of the talus bone is displaced medially and distal from the navicular bone. As a result, the plantar calcaneo-navicular ligament (spring ligament) and the tendon of the tibialis posterior muscle are stretched to the extent that the individual with pes planus loses the function of the medial longitudinal arch (MLA).

If the MLA is absent or non-functional in both the seated and standing positions, the individual has a condition called “rigid” flat feet. If the MLA is present and functional while the individual is sitting or standing up on their toes, but this arch disappears when assuming a foot-flat stance, the individual has “supple” or “flexible” flat feet. This condition is often treated with arch supports.

Flat feet can be genetic, and can also develop due to injury, illness, unusual or prolonged stress to the foot, faulty biomechanics, or as part of the normal aging process. Known risk factors include obesity and diabetes. If developed by adulthood, flat feet generally remain flat permanently.

Most flexible flat feet are asymptomatic, and do not cause pain. However, a person with flat feet often tends to overpronate when running. A runner who overpronates may be more susceptible to shin splints, back problems, and tendonitis in the lower limb. Running in shoes with extra medial support or using orthoses, may help to reduce pronation and may reduce risk of injury.

However, in some adults with flat feet, the tendon of the tibialis posterior muscle may be dysfunctional and lead to disabling weight bearing symptoms. While barefoot, these subjects activate additional lower leg muscles to complete an exercise that resists foot adduction. However, when the same subjects perform the exercise while wearing arch supporting orthotics and shoes, the tibialis posterior tends to be selectively activated. This suggests that the use of shoes with properly fitting, arch-supporting orthoses will enhance selective activation of the tibialis posterior muscle and can act as an adequate treatment for the undesirable symptoms of flat feet. (Kulig, K. et al (2005) Med. Sci Sports Exercise 37(1):24-29)

Rigid flat feet often indicate a significant problem in the bones of the affected feet, and can cause pain. Other flat feet related conditions, such as various forms of tarsal coalition (that is, two or more bones in the midfoot or hindfoot being abnormally joined) or an accessory navicular (which is an extra bone on the inner side of the foot) are typically diagnosed in childhood. If these conditions are diagnosed before the child's bone structure firms up permanently as a young adult, remedial action may be attempted. Rheumatoid arthritis can destroy tendons in the foot (or both feet) which can cause this condition, and if left untreated it can result in deformity and early onset of osteoarthritis of the joint. Such a condition can cause severe pain and considerably reduced ability to walk, even with orthoses.

Treatment of flat feet may also be appropriate if there is associated foot or lower leg pain, or if the condition affects the knees or the lower back. In the past, treatment has included foot exercises. In cases of severe flat feet, gradually modified orthoses have been used. The treatment includes gradually increasing the amount of material in the orthosis, augmenting the arched shape over the course of several weeks. The foot structure is thus gradually adjusted, giving the patient time to get used to the sensation of wearing orthoses.

Some patients resort to surgery. For example, one type of surgical intervention involves insertion of an implant into the sinus tarsi to prevents the calcaneus and talus from sliding relative to each other. This prevents the sinus tarsi from collapsing and thus prevents the arch from falling.

There is an ongoing need for better and more convenient orthoses for subjects having foot conditions, particularly but not limited to having flat feet.

### SUMMARY

An object of the present invention is to provide an improved device for alleviating at least one of the symptoms of a foot condition, such as flat feet. A further object of the embodiments described herein to overcome or alleviate at least one of the above noted drawbacks of related art systems or to at least provide a useful alternative to related art systems.

In its broadest form, the present invention provides an orthosis for footwear comprising an insert having a plurality of retainers, and a plurality of adjustment elements of predetermined configuration which can be removably retained by the insert to alter the shape of the insert adjacent the arch of a subject's foot when located in the footwear. In another aspect, the present invention provides an orthosis for footwear comprising;
an insert comprising a proximal portion, medial portion and distal portion and having an upper foot-facing surface, and a lower sole-facing surface, the insert defined by a periphery having a medial longitudinal edge and a lateral longitudinal edge,
one or more, preferably a plurality, of retainers located along adjacent the edge of the insert, preferably the medial longitudinal edge of the insert and/or the lateral longitudinal edges of the insert, and
one or more, preferably a plurality of adjustment elements removably located in respective retainers, wherein each adjustment element holds an adjacent portion of the lower surface of the insert at a predetermined angle relative to the footwear sole.

Typically, each adjustment element includes a generally wedge-shaped body, having 2 triangular faces and 3 quadrilateral faces. Each wedge has a pair of faces which meet at a predetermined angle from 5 to 25°, typically, 5°, 10°, 15° and 20°.

The adjustment element may be removably attached to the retainer by any convenient means such as interference fit, friction fit or snap fit. Typically, an adjustment element is at least partly inserted into a corresponding retainer means. The adjustment element may further include a means for removably locating the adjustable element in a respective retainer, such as a lug, flange or recess.

In one embodiment, the retainers are located adjacent the medial longitudinal edge of the insert, the medial edge being the edge which would normally be closest to the other foot, and thus adjacent the part of the foot sole that would normally exhibit an arch. This would typically be used for a subject having flat feet.

In another embodiment, the retainers are located adjacent the lateral longitudinal edge of the insert. This would typically be used for a subject a high arched foot posture.

The orthosis of the present invention can be customised to the subject's foot by including adjustment elements having different predetermined shapes, such as wedges having different angles. An adjustment element having a first wedge angle can be swapped for an adjustment element having a second wedge angle in response to changes to the subject's foot condition such as flat feet or a high arched foot. Furthermore, in cases of severe flat feet the orthoses can be gradually modified over time by periodically changing the adjustment element. For example, over the course of several weeks, the wedge angles in the orthosis can be slowly increased to raise the insert in the areas adjacent the foot arch. The foot structure may thus be gradually adjusted, giving the subject time to get used to the sensation of wearing orthoses, or changing the orthosis in response to changes in the subject's foot condition.

In use, the lower surface of the insert extends along at least part of the length of the sole of the subject's footwear and concomitantly, the upper surface extends along at least part of the length of the subject's foot. The insert comprises a generally planar distal portion, a proximal portion and a medial portion intermediate the distal portion and the proximal portion. In use, the distal portion is adjacent the ball of the foot and may optionally extend to the region of the toes. The proximal portion is adjacent the heel of the foot and is preferably shaped to form a cup that corresponds generally to the shape of the subject's heel. The medial portion is adjacent the mid-region of the foot.

Typically, the insert is removably located in an item of footwear. Alternatively, the insert is integral with the item of footwear. The insert and adjustment element can be made of any plastic material that can be shaped such as, for example polypropylene, polyvinylchloride, polyethylene terephthalate, or other copolymer material. The insert typically comprises a shell and over moulded innersole that together form a unitary integral orthotic insert.

The insert shell is preferably injection moulded from a polyolefin chosen from the polypropylene/polyethylene (PP/PE) family of polymers and comprises a number of shaped segments with voids therebetween. The innersole is preferably composed of styrene ethylene butylene styrene (SEBS). The SEBS material is preferably over moulded to form a complete innersole spanning across the top of the segment voids to create a transitional elastic web of material that allows different adjustment elements to be retained to create a variety of incrementally varying arch support profiles.

The shape of the lower, sole-facing surface of the orthotic typically corresponds to the shape of most standard shoe soles to allow for appropriate fitting of the device. Retainers, on the lower sole-facing surface of the orthotic allow for intimate locking of the adjustment elements into the device to provide varying degrees of support. Preferably each retainer is a groove or recess, and each adjustment element is substantially wedge shaped.

In another aspect of embodiments described herein there is provided a method of treating at least one symptom of a foot condition such as flat feet using the orthosis of the present invention comprising the steps of;
removably locating a plurality of adjustment elements in respective retainers in the insert,
locating the insert in an item of footwear, wherein each adjustment element holds the lower surface of the insert adjacent the element at a predetermined angle relative to the footwear sole.

The present invention may be provided in kit form, including left foot and/or right foot inserts and a plurality of adjustment elements having wedges of various angles. Other aspects and preferred forms are disclosed in the specification and/or defined in the appended claims, forming a part of the description of the invention.

In essence, embodiments of the present invention stem from the realization that modifying the angles in small areas of a shoe insert corresponding to different areas on the users foot can be used to customise the shape of an insert to a subject's foot in a manner that relieves foot conditions or changes the alignment of the foot such as but not limited to flat feet.

Advantages provided by the present invention comprise the following:
Improved control over the amount of orthosis adjustment,
Ease of orthosis adjustment,
Provision of a customised orthosis using mass produced components,
Ease of manufacture,
Providing a more solid deeper heel support to ensure that the foot more accurately the rearfoot is held on the device.
Provide manipulation of the alignment of the heel or rearfoot angle without the heel moving off the orthotic supported by means of the deep heel cup.

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### BRIEF DESCRIPTION OF THE DRAWINGS

Further disclosure, objects, advantages and aspects of preferred and other embodiments of the present application may be better understood by those skilled in the relevant art by reference to the following description of embodiments taken in conjunction with the accompanying drawings, which are given by way of illustration only, and thus are not limitative of the disclosure herein, and in which:

FIG. 1 illustrates the bones of the foot as viewed from above, looking down (FIG. 1a) and from the side (FIG. 1b);

FIG. 2 illustrates a side view of an insert of the present invention without adjustment elements;

FIG. 3 illustrates a side view of the insert of FIG. 1 with adjustment elements inserted along the longitudinal medial edge of the insert

FIG. 4 illustrates a bottom view of left foot and right foot versions of the insert and adjustment elements viewed looking towards the shoe sole;

FIG. 5 illustrates the embodiment of the invention shown in FIG. 3 viewed looking towards the shoe upper;

FIG. 6 illustrates the embodiment of the invention shown in FIG. 3 viewed looking from the shoe heel with adjustment elements in the form of wedges for a user having flat feet;

FIG. 7 illustrates the embodiment of the invention shown in FIG. 3 viewed looking from the shoe heel with adjustment elements for a user having a high arched foot posture;

FIG. 8 illustrates the embodiment of the invention shown in FIG. 3 in perspective view looking from the shoe heel;

FIG. 9 illustrates one embodiment of the adjustment elements according to the present invention in perspective view (FIG. 9a), side view (FIG. 9b) and bottom view (FIG. 9c) and another embodiment in side view (FIG. 9d) and bottom view (FIG. 9e); and

FIG. 10 illustrates four embodiments of the adjustment elements shown in FIG. 9 having a wedge-shaped body with two faces at an angle of 5° (FIG. 10a), 10° (FIG. 10b), 15° (FIGS. 10c) and 20° (FIG. 10d).

### TABLE OF PARTS

| | | | |
|---|---|---|---|
| 1-5 | Metatarsals | 6 | Phalanges (toe bones) |
| 7 | Cuboid | 8 | Lateral cuneiform |
| 9 | Intermediate cuneiform | 10 | Medial cuneiform |
| 11 | Calcaneus | 12 | Trochlea of talus |
| 13 | Transverse tarsal joint | 14 | Tarsometatarsal joint |
| 15 | Calcaneus (heel bone) | 16 | Head of talus |
| 17 | Navicular | 18 | Sesamoid |
| | | 20 | Insert |
| | | 22 | Proximal portion |
| 23 | Medial portion | 24 | Distal portion |
| 25 | Upper surface | 26 | Lower surface |
| | | 28 | Medial longitudinal edge |
| 29 | Lateral longitudinal edge | 30 | Adjustment elements |
| | | 32 | Retaining means |
| 33 | Shoe | 34 | Heel cup |
| 35 | Body of Adjustment elements | 36 | Lug |
| 37 | Face | 39 | Groove |

### DETAILED DESCRIPTION

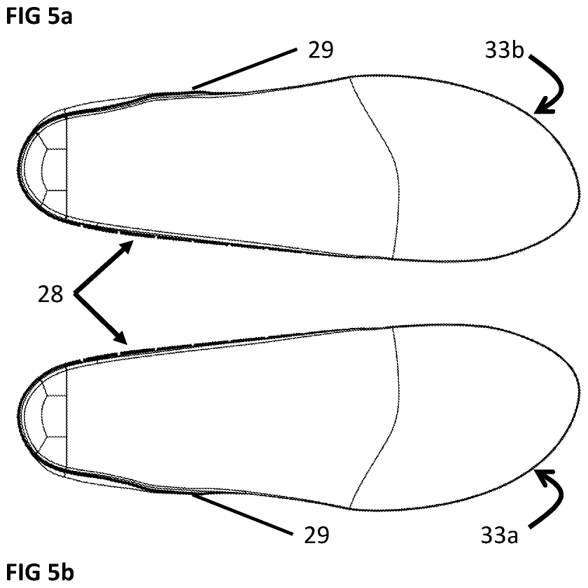

For purposes of description herein, the terms “upper,” “lower,” “right,” “left,” “rear,” “front,” “vertical,” “horizontal,” “interior,” “exterior,” “lateral”, “medial” and derivatives thereof shall relate to the invention as oriented in FIG. 5. However, it is to be understood that the invention may assume various alternative orientations, except where expressly specified to the contrary. It is also to be understood that the specific devices and processes illustrated in the attached drawing and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise. Additionally, unless otherwise specified, it is to be understood that discussion of a particular feature of component extending in or along a given direction or the like does not mean that the feature or component follows a straight line or axis in such a direction or that it only extends in such direction or on such a plane without other directional components or deviations, unless otherwise specified.

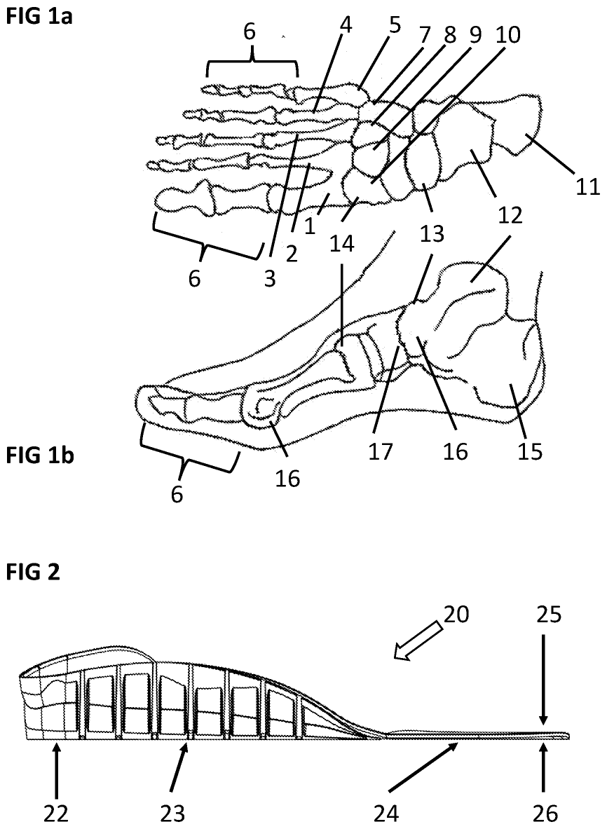

FIG. 1 illustrates the bones of the foot viewed from the top (FIG. 1a) and from the side (FIG. 1b).

FIG. 2 illustrates a side view, towards the medial longitudinal edge (28) of an insert (20) of the present invention for a left foot. The illustration does not depict the adjustment elements. The insert (20) comprises a proximal portion (22), medial portion (23) and distal portion (24). In use, the upper surface (25) of the insert (20) faces the subject's foot, while the lower surface (26) faces the sole of the footwear. The insert (20) is defined by a periphery having a medial longitudinal edge (28) and a lateral longitudinal edge (29, not shown in this view).

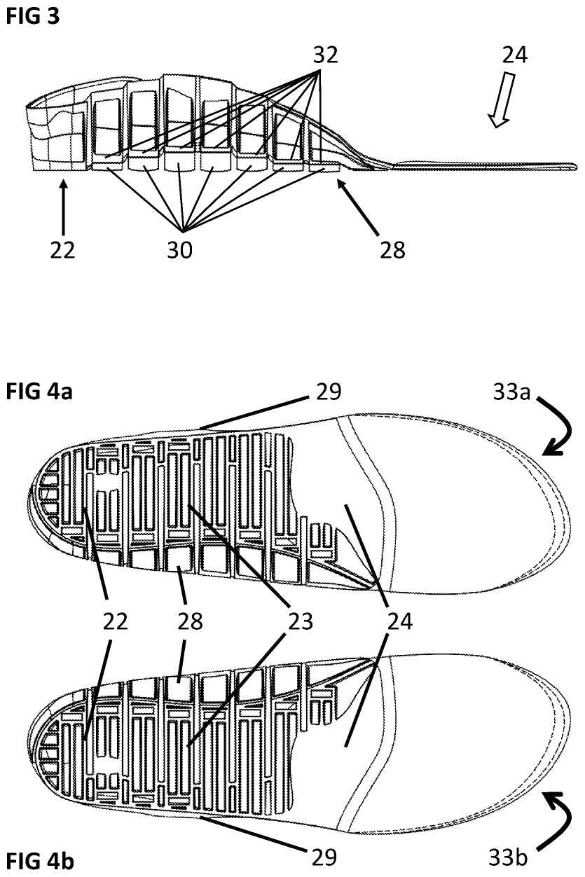

FIG. 3 illustrates a side view of the insert (20) of FIG. 2 with adjustment elements (30) inserted in retaining means (32) located in the medial portion (23) and adjacent the medial longitudinal edge (28) of the insert (20).

FIG. 4 illustrates left foot (FIG. 4a) and right foot (FIG. 4b) versions of the insert (20) located in a right shoe (33a) and left shoe (33b), the outline of which are shown in broken lines). The insert is viewed looking towards the sole of the shoes (33). In this view the medial longitudinal edge (28) and lateral longitudinal edge (29) of the inserts (20) can be easily seen.

Each individual adjustment element in the orthotic allows for modification of different segments of the device depending on the need of the user.

The lower surface of the insert (20) comprises multiple segments with voids therebetween, creating a pattern which is adapted to be specific to the functional requirements. The segments are created to impart strength and adequate support where it is required by the foot of the user. The ‘E’ shape profile on the edge of each segment is to allow the adjustment elements (30), preferably in the form of wedges, to be readily pushed in with a low frictional resistance whilst applying sufficient force to hold the wedges securely. The wall section at the base of each segment is minimised to allow easy flex under the influence of the wedge.

FIG. 5 illustrates the embodiment of the invention shown in FIG. 4 viewed looking towards the upper of the shoes (33a, 33b).

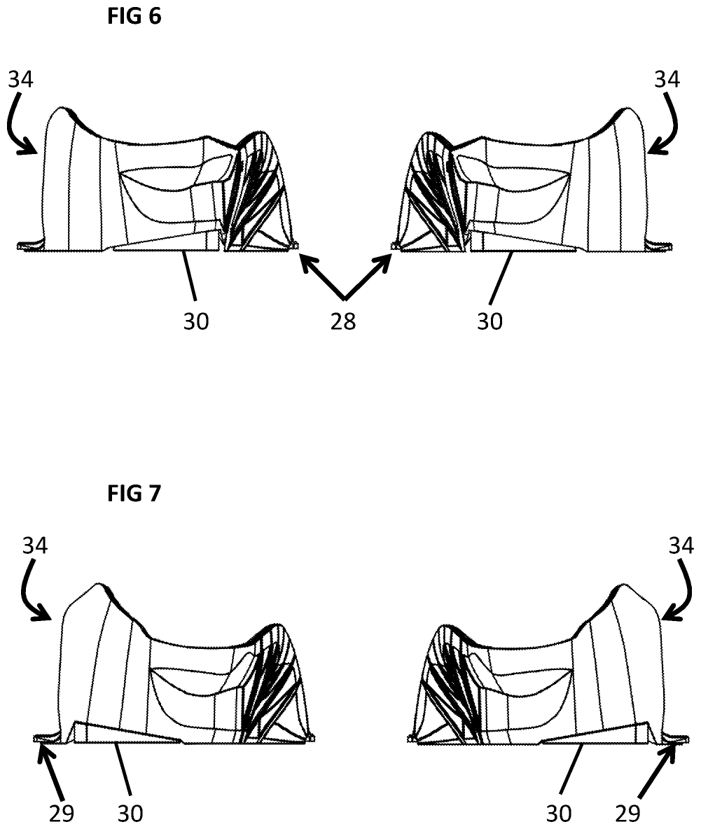

FIG. 6 illustrates the embodiment of the invention shown in FIG. 4 showing a left and right insert (20) viewed looking from the heel cup (34) towards the toe with showing adjustment elements (30) in the form of wedges adjacent the medial longitudinal edge (28) of the insert (20) for a user having flat feet.

FIG. 7 illustrates the embodiment of the invention shown in FIG. 4 viewed looking from the shoe heel with adjustment elements (30) in the vicinity of the lateral longitudinal edge (29) of the insert (20) for a user having a high arched foot posture.

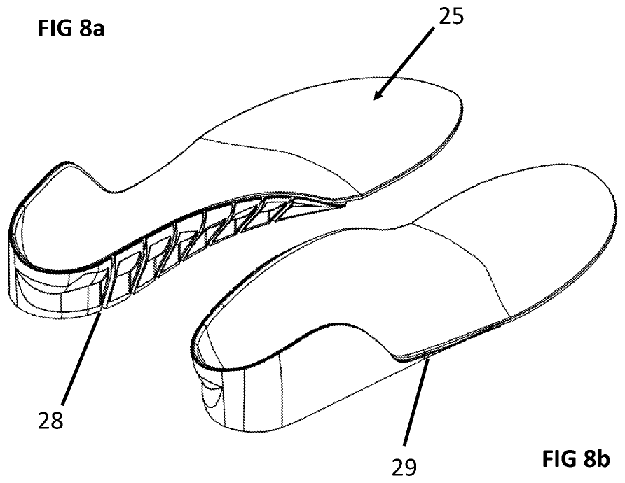

FIG. 8 illustrates the embodiment of the invention shown in FIG. 4 with respect to an insert (20) for a left shoe (33a, not shown) and a right shoe (33b, not shown) in perspective view looking from the proximal portion (22) towards the distal portion (24) across the upper surface (25).

The insert (20) includes a generally planar distal portion (24), a proximal portion (22) and a medial portion (23) interconnecting the distal portion (24) and the proximal portion (22). The distal portion (24) is located adjacent the ball of the foot, that is the area adjacent the sesamoid bone (18) and may optionally extend across at least part of the phalanges (6) (toe bones). The proximal portion (22) supports the calcaneus (11) (heel) of the foot and has a centrally disposed concave depression and a generally planar border located posteriorly with respect to the concave depression which together form the heel cup (34). The heel cup (34) produces a medial and lateral wedging effect against the heel of the foot. This helps to aid positioning of the heel and aligning the heel bone with the ankle and lower leg muscles.

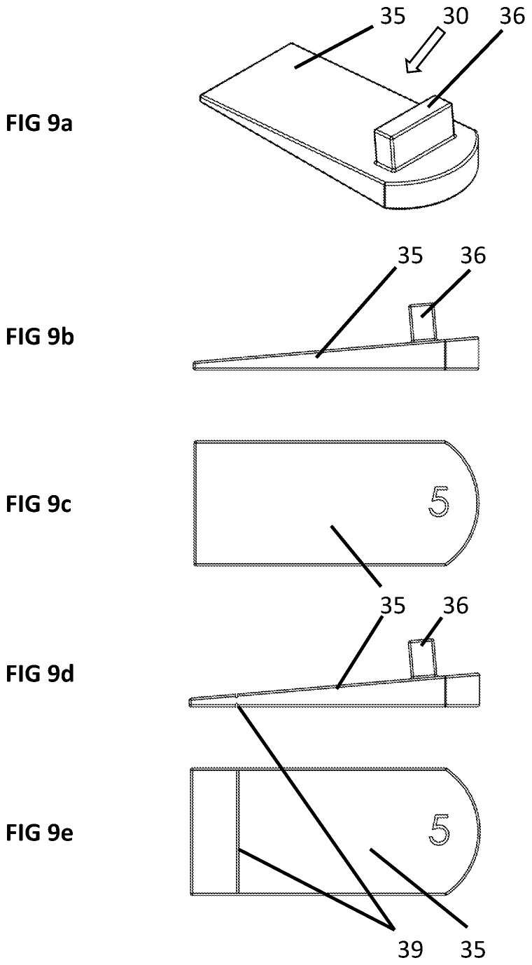

FIG. 9 illustrates one embodiment of the adjustment elements (30) according to the present invention shown in perspective view (FIG. 9a), side view (FIG. 9b) and bottom view (FIG. 9c). This embodiment includes a generally wedge-shaped body (35) and a lug (36) to facilitate retention in the retainer of the insert (20). The wedge-shaped body (35) has 5 faces (37) 2 triangular faces and 2 flat quadrilateral faces and a curved face. The wedge has a pair of faces (37a, 37b) which meet at a predetermined angle of 5°.

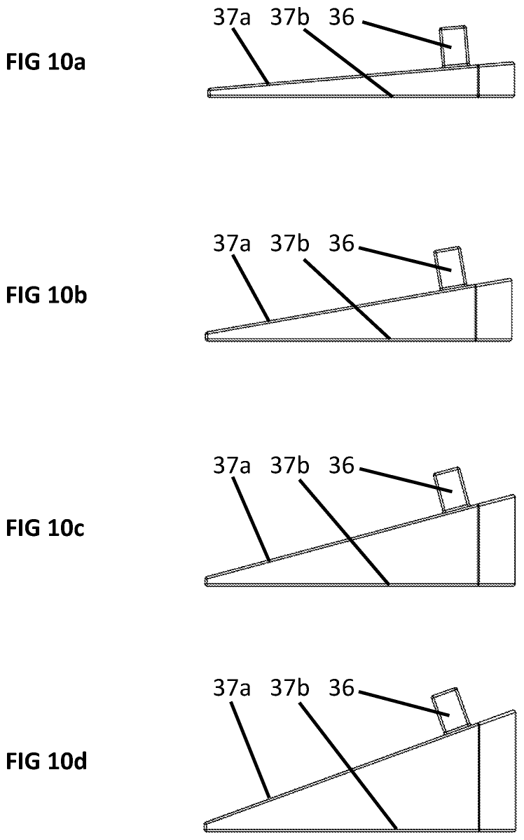

The orthosis of the present invention can be customised to the subject's foot by using these adjustment elements (30) in the form of wedges having different angles. FIG. 10 illustrates four embodiments of the adjustment elements shown in FIG. 9 having a wedge-shaped body with two faces (37a, 37b) at an angle of 5° (FIG. 10a), 10° (FIG. 10b), 15° (FIGS. 10c) and 20° (FIG. 10d). An adjustment element having a first wedge angle of 5° can be swapped at a later date for an adjustment element having a second wedge angle of 10°, 15° or 20° in response to changes to the subject's foot condition such as flat feet or a high arched foot. In cases of severe flat feet, the adjustment elements (30) can be gradually modified over time. For example, the wedge angle can be slowly increased over the course of several weeks to raise the insert (20) in the areas adjacent the foot arch.

FIG. 9 also illustrates another embodiment of the adjustment elements (30) according to the present invention, shown in side view (FIG. 9d) and bottom view (FIG. 9e). Again, this embodiment includes a generally wedge-shaped body (35) and a lug (36) to facilitate retention in the retainer of the insert (20). It is substantially the same as the embodiment depicted in FIGS. 9a to 9d except for a groove (39) providing a weakened portion in the faces (37a, 37b) of the wedge-shaped body (35). The wedge-shaped body (35) may be ruptured or cut along the groove (39) to alter the length of the adjustment element.

The medial portion supports the mid-region of the foot. The medial portion may include a centrally disposed, generally planar raised portion, a first depression for supporting the medial arch of the foot, a second depression for supporting the lateral arch of the foot, and a third depression for supporting the metatarsal arch of the foot.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, means-plus-function clauses are intended to cover structures as performing the defined function and not only structural equivalents, but also equivalent structures.

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

1. An orthosis for footwear comprising an insert having a plurality of retainers, and a plurality of adjustment elements of predetermined configuration, wherein each of the adjustment elements may be positioned on a lower sole-facing surface of the orthosis, and wherein the adjustment elements are configured to be removably retained by the insert to alter a shape of the insert adjacent an arch of a subject's foot when located in the footwear.

2. The orthosis for footwear of claim 1 comprising;
the insert comprising a proximal portion, medial portion and distal portion and having an upper foot-facing surface, and a lower sole-facing surface, the insert defined by a periphery having a medial longitudinal edge and a lateral longitudinal edge,
the retainers located adjacent the edge of the insert, and
the adjustment elements removably located in respective retainers, wherein each of the adjustment elements holds an adjacent portion of the lower surface of the insert at a predetermined angle relative to the footwear sole.

3. The orthosis according to claim 1 wherein each of the adjustment elements includes a generally wedge-shaped body.

4. The orthosis according to claim 3 wherein each of the adjustment elements has 2 triangular faces and 3 quadrilateral faces, at least one pair of the faces meeting a predetermined angle from 5° to 25°.

5. The orthosis according to claim 4, wherein the predetermined angle is 5°, 10°, 15° or 25°.

6. The orthosis according to claim 1 wherein each of the adjustment elements is removably attached to the retainer by one or more of an interference fit, friction fit or snap fit.

7. A method of treating at least one symptom of a foot condition using an orthosis comprising:
an insert comprising a proximal portion, medial portion and distal portion and having an upper foot-facing surface, and a lower sole-facing surface, the insert defined by a periphery having a medial longitudinal edge and a lateral longitudinal edge,
locating a retainer along an adjacent the edge of the insert, and
an adjustment element holding an adjacent portion of the lower surface of the insert at a predetermined angle relative to a footwear sole;
removably locating the adjustment element in the retainer in the insert, and
locating the insert in an item of footwear.

8. The method according to claim 7 wherein the insert is located in the item of footwear such that the upper foot-facing surface extends along at least part of the length of a subject's foot, the distal portion being adjacent a ball of the subject's foot, the proximal portion being adjacent a heel of the subject's foot and the medial portion being adjacent a mid-region of the subject's foot.

9. The method according to claim 7 wherein the method further comprises periodically changing the adjustment element over a period of time.

10. The method according to claim 1, wherein the foot condition is flat feet.

11. The orthosis according to claim 2, wherein each of the adjustment elements includes a generally wedge-shaped body.

12. The orthosis according to claim 6, wherein each of the adjustment elements has 2 triangular faces and 3 quadrilateral faces, at least one pair of the faces meeting a predetermined angle from 5° to 25°.

13. The orthosis according to claim 12, wherein the predetermined angle is 5°, 10°, 15° or 25°.

14. The orthosis according to claim 2, wherein each of the adjustment elements is removably attached to the retainer by one or more of an interference fit, friction fit or snap fit.

15. The method according to claim 2, wherein the foot condition is flat feet.
